(19) United States
(12) Patent Application Publication
(10) Pub. No.: US 2004/0033557 A1
(43) Pub. Date: Feb. 19, 2004

(54) Method of generating and recovering gas from subsurface formations of coal, carbonaceous shale and organic-rich shales

(76) Inventors: Andrew R. Scott, Austin, TX (US); Joe E. Guyer, Houston, TX (US)

Correspondence Address: MORRISON & FOERSTER LLP, 1650 TYSONS BOULEVARD, SUITE 300, MCLEAN, VA, 22102, US

(21) Appl. No.: 10/640,273

(22) Filed: Aug. 14, 2003

Related U.S. Application Data
(63) Continuation of application No. 09/696,304, filed on Oct. 26, 2000, now abandoned.

(51) Int. Cl. C12P 1/04; C12P 39/00

(52) U.S. Cl. 435/42; 435/170; 435/132

(57) ABSTRACT

A method of generating and recovering gas from naturally existing subsurface formations Of coal, carbonaceous shale or organic-rich shales comprising the steps Of: injecting into fracture of the subsurface formation, under substantially anaerobic conditions, a consortia of selected anaerobic, biological microorganisms for in situ conversion of organic compounds in said formation into methane and other compounds; and producing methane through at least one well extending from said subsurface formation to the surfaces.

### BACKGROUND OF THE INVENTION

1. Field of the Invention

The present invention pertains to methods of producing gas from subsurface formations. More specifically, the present invention pertains to methods of recovering gas from naturally existing; subsurface formations of coal, carbonaceous shale and organic-ri.ch shales through in situ conversion of organic compounds into methane and other compounds by consortia of selected anaerobic biological microorganisms.

2. Description of the Prior Art

Coal gas and shale gas production are increasingly an important energy source for the United States and the rest of the world. Annual coal bed methane production in the United States has increased from less than 85 Bcf in 1985 to over 1,100 Tcf in 1998 and now accounts for more than seven percent of nonassociated gas reserves in the United States. Therefore, coal bed methane represents an important energy source. A significant amount of natural produced from coal beds, carbonaceous shales and organic rich shales is secondary biogenic methane that formed under natural processes after burial, coalification and subsequent uplift and cooling. In both Coal bed and shale reservoirs the majority of the gases are sorbed on the microporous matrix of the organic fraction of the rock, whereas relatively minor amounts of gas are sorbed on the inorganic part of the rock. The amount of gas sorbed to the organic, matter increases with increasing pressure until the surface of the organic matter is covered by a monolayer of gas molecules at which time no more gas can be sorbed to the organic matter. The coal or shale becomes saturated with respect to methane—once the monolayer capacity has been reached. Coal and shale gas are produced by lowering reservoir pressure, resulting in desoption of gases from the surface of organic mater and diffusion of gases through the organic matrix towards natural fractures or cleats. During production, gases desorbed from the microporous coal matrix diffuse toward naturally occurring fractures and cleats and/or induced fractures in the reservoir and migrate to the wellbore following Darcy's Law. Methane is the dominant gas produced, but other gases including carbon dioxide, ethane, propane, butane, and hydrogen, as well as oil may be produced in varying proportions.

Most coal beds are stimulated prior to production following standard industry practices of reservoir hydrofracturing, but may also include open hole cavity completions. Hydrofracture stimulation is the most common practice, but care must be used in selecting fluids that come in contact with the reservoir because the coal may react adversely with the stimulation fluids. During open-hole cavity completions, the reservoir is pressurized and then suddenly depressurized causing the friable coal to slough off into the wellbore and be carried to the surface, thereby creating a large cavity and fracture systems in the subsurface. Open-hole cavity completions appear only to work in a selected area of the San Juan Basin in Colorado and New Mexico. Coal beds in other basins in the United States and other parts of the world are generally stimulated using fracture techniques rather than openhole cavity completions. Drilling wells horizontally into coal beds has also been attempted, but the results of this technology to date are mixed. Regardless of the techniques employed coal gas and shale gas production by prior art methods is limited.

The use of bacteria to enhance oil production is known. For example, U.S. Pat. No. 3,185,216 discusses injection of bacteria into saline formation waters which are in contact with oil. Bacteria injected into formation water migrate to the oil water contact where they metabolize the oil and create byproducts that enhance the mobility of the reservoired oil. Injection of microorganisms into the water rather than directly into the oil prevents the bacteria from multiplying too quickly, which would reduce permeability and prevent bacterial access to deeper parts of the reservoir.

Biological conversion of coal to methane in underground coal mines and cavities, shallow peat bogs, and surface bioconversion in bioreactors or vessels is also known. U.S. Pat. No. 3,640,846 discusses the conversion of a mixture of coal and sewage sludge containing ‘methane-producing anaerobic bacteria. This reaction would take place in vessels or bioreactors although the bacteria could he injected with water into abandoned underground coal mines. This process is assumed to work best in lower rank coals, such as lignite and subbituminous, because they contain appreciable cellulose like material upon which the bacteria may feed. This process was designed to be dependent upon the use of a vessel from which air is excluded, and therefore, does not apply to in situ biological conversion of coal to methane or other compounds.

U.S. Pat. No. 4,358,537 describes a process for the biological in situ preparation and conversion of peat to useful products that can be subsequently converted to hydrocarbon fuels using a bioreactor. This process utilizes aerobic bacteria to prepare the peat and to remove oxygen from the system so that anaerobic bacteria can convert peat to useful products. It is noted that natural peat deposits are generally located at or near the surface so that liquids containing bacteria and nutrients are introduced at the surface or at very shallow depths. The resulting mixture of bioconverted compounds that include hydrolysis products released from the peat are pumped from the peat to active anaerobic digesters located at the surface where methane and other useful products are produced. it is recommended that sewage sludge from treatment plants, lake or river water, or recycled fluids from biological. reactors be utilized in this process to covert carbonaceous materials removed from the peat into gaseous or liquid hydrocarbon fuels. This process is only applicable to shallow peat deposits and involves the use of a bioreactor to generate useful byproducts.

In U.S. Pat. Nos. 4,846,769 and 4,826,769 microorganisms are introduced into a subterranean cavern or salt formation, solution-mined limestone caverns and/or caverns physically mined in limestone or granite, where controlled conditions are maintained. This method is particularly adaptable to mediated bioconversion, which requires large reactors, and requires that the subterranean cavity be free of fractures. Coal is initially crushed and pretreated with an alkaline solution in the reactor at high temperatures, with or without oxygen. The reactor is then cooled using heat exchangers, to temperatures favorable for conducting the fermentation of coal, peat, lignite, subbituminous coal, bituminous coal, anthracite coal, into methane. The method may be performed in the cavity as a continuously stirred tank reaction, or plug flow or as a staged reaction. Gases generated during the process are removed from the upper part of the cavern by means of a fan or compressor, whereas liquids or solid materials are removed by pumping from appropriate depths within the cavity.

U.S. Pat. No. 5,424,195, describes a method and apparatus for biological conversion of coal to methane using a consortium of microorganisms capable of degrading the coal into methane under certain conditions. Unlike U.S. Pat. Nos. 4,846,769 and 4,826,769 in which a coal substrate is added to a cavity, this patent refers to injection into an abandoned coal mine in which the cavity provides the feedstock for the bacteria. As described in the patent, microorganisms were transported in household sewage from septic tanks to adjacent abandoned coal mines. Over time, the entrances of abandoned coal mines continued to subside or be sealed thereby creating an anaerobic environment for the bacteria. The bacteria introduced in this manner adapt over a period of time (up to several decades) so that they could convert coal into methane and other gases. This art could be applied to other undiscovered abandoned mine sites in which sewage was initially introduced into the mine, and bacterial degradation of the coal had occurred after the sewage ceased entering the mine. If the coal seam, or coal mine (which may contain one or more coal seams) contains bacteria derived by the methods described above, then nutrients would be pumped into the coal-containing substrate and the in situ conditions adjusted to promote the conversion of coal into methane. over time, the coal pillars supporting the root would collapse thereby exposing fresh coal surfaces which could be microbially degraded by the microorganisms. The gas produced through the bioconverersion of coal into methane would accumulate at high points in a mine and be recovered by means of a gaseous pipe leading from the mine ceiling to a recovery tank. This process differs from previous art because the bacteria are initially derived from household sewage entering an abandoned coal mine and the process occurs at much lower temperatures and pressures common in abandoned coal mines.

While the prior art does disclose various methods of utilizing biological microorganisms for converting some forms of coal to gas, none of them disclose methods for economical, recovery of gas from naturally existing subsurface formations of coal, carbonaceous shale and organic-rich shale by in situ conversion of such materials through consortiums of microorganisms injected into the formation. Such a process should be well received by the energy industry.

### SUMMARY OF THE PRESENT INVENTION

If only one-hundredth of one percent of the coal in the United States were converted into methane using miorobially enhanced gas generation, then gas reserves in the United States would increase by 23 Tcf or sixteen percent of current nonassociated reserves. The method of the present invention can be applied to coals, carbonaceous shales, organic-rich shales or shales at any level of thermal ‘maturity, temperature, and depth, and in the presence of fresh or highly saline formation waters. This is possible because modification and adjustment of the bacterial consortia arid/or nutrients with the present invention maximizes the bacterial degradation of the organic matter and subsequent generation of methane, hydrogen, carbon dioxide, and other gases. conversion of a higher percentage of coal, carbonaceous shales, and/or organic-rich shales into methane would significantly increase natural gas reserves, thereby providing a stable, economically favorable, and environmentally clean energy source for the United States and many other parts of the world.

Unlike previous art, which uses surface bioreactors, abandoned coal nines or other cavities as subterranean bioreactors at relatively shallow depths and lower pressures, the present invention involves the injection of bacteria and nutrients, under pressure, into naturally occurring fractures or cleats in coal beds as well as fractures induced during stimulation of coal bed ‘methane shale gas wells. The nutrients may include, bat are not limited to, carbon dioxide which may be sequestered into the coal bed prior to injection of bacteria and other nutrients. Nutrients may include both trace elements, organic compounds (such as molasses), and solutions containing ions that will promote and/or modify bacterial growth, These ion s may include but are not limited to calcium, magnesium, iron, sodium, potassium, sulfate, and bicarbonate. Additionally, the present invention may also be applied to carbonaceous shales, organic-rich shales, and shales that have naturally occurring or artificially induced fractures and contain sufficient organic matter to support bioconversion. The bacterial consortia are injected under pressure into the reservoir where they metabolize and convert the organic matter along the surface of fractures and/or cleats into methane, hydrogen and other useful products.

The progressive increase of carbon dioxide in the atmosphere and the potential of global warming has prompted the United States and other countries to reduce carbon dioxide emission through the sequestration of carbon dioxide in coal beds, abandoned oil reservoirs, saline aquifers, and in deep oceans. The close proximity of unmined and unmineable coal to coal-fired power plants makes sequestration of carbon dioxide in coal beds favorable, particularly if sequestering carbon dioxide removes methane from the coal in the process. The economic benefit of removing methane from coal beds using carbon dioxide will lower the overall cost of carbon dioxide sequestration, thereby benefiting the electricity consumer who ultimately will pay for carbon dioxide sequestration. The present invention potentially adds an additional economic benefit through the in situ bioconversion of greenhouse gases, specifically carbon dioxide and carbon monoxide, sequestered into coal beds, into methane or other useful organic compounds. The carbon dioxide and carbon monoxide are initially injected into coal beds where they are sorbed onto coal surfaces and/or dissolved into formation water. Bacteria and nutrients are injected into the coal beds where the bacterial consortia convert the sequestered carbon dioxide into methane and other compounds.

The process of bioconversion of organic matter into methane involves a bacterial consortium that breaks down the organic matter in coal or shale into simple organic compounds that can be utilized by methanogens. During carbonate reduction, bacterial consortia utilize carbon dioxide that is sorbed on the coal matrix or dissolved as bicarbonate in formation water as a carbon source and derive three of the four hydrogen atoms used to form methane from formation water, in this application of the present invention carbon dioxide derived from a variety of outside sources including coal-fired power plants, would be sequestered in coal beds and bacterial consortia and nutrients would be added to the coal beds. The bacteria and nutrients would be adjusted to encourage the bioconversion of sequestered carbon dioxide into methane and other useful products. As carbon dioxide and other organic matter are metabolized, sorption sites on the organic matter become available for the methane molecules. The process of carbon dioxide sequestration followed by bioconversion of the carbon dioxide into methane can be repeated, thereby contributing to the reduction of greenhouse gas emissions as well as providing environmentally clean fuels such as methane.

The present invention differs from each of the prior art methods described above in several areas including the injection of bacterial consortia and nutrients under pressure into naturally occurring cleats and/or fractures as well as artificially generated hydrofractures rather than simple bioconversion in cavities or underground mines, and also provides a means for the conversion of sequestered carbon dioxide into methane. Additionally, the present invention is not limited to coal beds, but rather expands the bioconversion process to other types of organic-rich sediments and includes the in situ bioconversion of sequestered carbon dioxide. The present invention utilizes several methods for injecting bacteria and nutrients into organic-rich sediments for recovering methane therefrom. Assuring that bacterial consortium and nutrients access the largest possible part of the fracture system is critical for the economic in situ bioconversion of organic matter into methane, hydrogen, and other useful products.

Many other objects and advantages of the invention will be understood from reading this description which follows, in conjunction with the accompanying drawings.

### BRIEF DESCRIPTION OF THE DRAWINGS

FIG. 1 is a flow chart illustrating a three-step process for bioconversion of organic matter to methane and other compounds by anaerobic microorganisms, according to preferred methods of the present invention;

FIG. 2 illustrates a biochemical reaction of various bacterial species in a consortium of microorganisms for bioconversion of coal and/or shales, according to preferred methods of the present invention;

FIG. 3 illustrates another biochemical reaction of other bacterial species in a consortium of Microorganisms for bioconversion of coal and/or shales, according to other preferred methods of the present invention;

FIG. 4 is an elevation view of drilling and support equipment for drilling a well, injecting bacteria and nutrients, and recovering gas from subsurface formations of coal and/or shale, according to preferred methods of the present invention;

FIG. 5 is a pictorial representation of a well drilled into a subsurface coal and/or shale formation for illustrating recovery of gas therefrom by bioconversion according to a preferred method of the present invention;

FIG. 6 is an isometric representation of a subsurface coal and/or shale formation illustrating an arrangement of injector wells and recovery wells drilled there into for recovering gas therefrom by bioconversion according to another preferred method of the present invention;

FIG. 7 is a plan view of the injector and recovery well arrangement of FIG. 6;

FIG. 8 is an isometric representation of a subsurface coal and/or shale formation illustrating another injector and recovery well arrangement for recovering gas by bioconversion according to another preferred method of the present invention;

FIG. 9 is a plan view of the injection and recovery well arrangement of FIG. 6;

FIG. 10 is an isometric representation of a subsurface coal and/or shale formation illustrating another injection and recovery well arrangement for recovering gas by bioconversion according to still another preferred method of the present invention in a first period of time; and

FIG. 10 is an isometric representation of the subsurface coal and/or shale formation of FIG. 9 illustrating injector and recovery well arrangements at a second and later period of time.

### DESCRIPTION OF PREFERRED EMBODIMENTS

Anaerobic bacterial consortia occur naturally in many coal beds and organic-rich shales and often are the dominant source of methane produced from the coals. Unlike the prior art, the present invention applies to in situ bioconversion of organic matter contained in coal beds, carbonaceous shales, organic-rich shales, and other sediments that contain sufficient organic matter to support bioconversion through the injection of bacteria and nutrients into naturally occurring or artificially induced fractures.

The present invention recognizes that methanogenic bacteria and other anaerobic bacterial consortia can exist under a much wider range of temperatures, pressures, pH conditions, and salinities than surface or shallow subsurface. The following table illustrate this fact.

| Parameter | Range |
|---|---|
| Oxygen | 10⁻⁵⁶ moles/liter |
| Temperature | 36° to 230° F. |
| Salinity | 23 to 69,000 ppm |
| pH | 3.0 to 9.2 |
| substrate | simple organic compounds |
| Hydrogen | 10⁻⁵ to 10⁻⁴ atm |

Therefore, the present invention can be applied to organic-rich sediments that occur in highly diverse natural settings. Naturally occurring bacteria that are isolated from organic-rich sediments, including peat, lignite and coal beds, carbonaceous shales, organic-rich shales, and shales at any level of thermal maturity, can be identified, cultured, and injected into naturally occurring or artificially induced fractures. Naturally occurring bacterial consortia obtained from the subsurface are presumed to have evolved through time by the process of natural selection to be highly efficient at metabolizing the organic-material from the sediments in which they were obtained. The bacteria comprising the consortia may also have approached an equilibrium condition in which the populations are relatively well balanced resulting in maximized bioconversion of organic matter.

Nutrients, trace elements, salinity, pressure, pH, and other environmental factors that affect bioconversion of coal samples collected from a specific organic-rich sediments (i.e. such as a specific coal bed, coal package within a basin) can be identified in the laboratory through experimentation. The bacterial consortia and nutrients, injected into the subsurface under anaerobic or partially aerobic conditions, are subsequently monitored and adjusted to maximize the in situ generation of methane and other economic gases. Alternatively, nutrients and bacteria derived from commercial sources can be injected into organic rich sediments in a similar manner. In some cases, a combination of naturally occurring bacterial consortia and commercial bacterial consortia may be injected into organic-rich sediments if the process maximizes methane or hydrogen production.

The present invention also includes the in situ bioconversion of carbon, dioxide and/or carbon monoxide sequestered in coal beds into methane and other useful compounds. Carbon dioxide and carbon monoxide can be considered as a nutrient source for anaerobic bacteria. Therefore, greenhouse gases such as carbon dioxide and/or carbon monoxide, derived directly or ‘Indirectly from outside sources such as coal-fired power plants, can be sequestered into coal beds and then converted to methane through in situ bioconversion. The carbon dioxide sequestered in coal beds is sorbed to the surface of the coal and/or dissolved as bicarbonate and other ions in formation waters that fill fractures or cleats. Anaerobic bacteria use the carbon dioxide as a food source and obtain most of the hydrogen required for methane generation from the formation water and/or organic compounds or moisture that are part of the coal structure. Therefore, the present invention offers a mechanism by which greenhouse gases, such as carbon dioxide and carbon monoxide, are first sequestered into subsurface coal beds rather than being emitted to the atmosphere and then are subsequently converted into methane and other compounds through in situ bioconversion.

The method of the present invention relates to the application of anaerobic microorganism and/or nutrients injected under anaerobic or predominantly anaerobic conditions into the subsurface to promote the in situ conversion of organic matter into useful products such as methane and hydrogen. The present invention can be applied to coal, shale, carbonaceous shale, and/or organic-rich shale in the subsurface. The nutrient sources may be injected before the addition of bacteria, simultaneously with the bacteria, or after the bacteria have been injected into the subsurface. Injection of the bacteria and nutrients occurs through naturally occurring fractures and/or artificially induced fractures rather than into subterranean cavities as in the prior art. Additionally, the present invention specifically encompasses the injection of carbon dioxide and/or carbon monoxide into coal beds followed by the injection of nutrients and/or bacteria; the bacteria will convert the sequestered carbon dioxide into methane and other useful compounds. In this case, carbon dioxide and carbon monoxide are considered to be food stock or nutrient sources for the bacteria. The interaction of bacterial consortia and the methodology and technology of injecting bacteria and/or nutrients into the subsurface remain the same. The importance of bacterial species interaction and the basic metabolic pathways of methanogens

Methane is generated from bacterial consortia under natural conditions when bacteria and/or nutrients are introduced into permeable coal beds or other organic-rich sediments by meteoric waters moving basinward from the outcrop. Biogenic methane generated under these conditions is termed secondary biogenic methane to distinguish it from primary biogenic methane that is formed in a peat swamp through the decay of organic material. After deposition, the organic matter in the coal and/or shales is buried and. undergoes thermal maturation. Upon uplift and thermal cooling, meteoric water enters the coal and/or shale bringing bacteria and nutrients into the subsurface and the bacteria metabolize the organic matters, including the organic compounds generated during thermal maturation. Methane associated with lower-rank. coals (i.e. lower thermal maturity levels) in basins such as the Powder River Basin may be entirely secondary biogenic, whereas higher-rank (i.e. higher levels of thermal maturity) coal basins may contain a mixture of thermogenic (i.e., gases generated during thermal maturation) and secondary biogenic gases. Based on isotopic analyses and hydrological evaluation, an estimated 2 Tcf of secondary biogenic gas has been produced from the San Juan Basin, suggesting that in situ bioconversion of Coal to Methane may be a significant source of energy.

Aerobic bacteria are capable of metabolizing a wide variety of organic substrates. Methanogens, however, are generally limited to hydrogen, carbon dioxide, and simple organic compounds, most of which contain only one atom. These simple-organic compounds include formate, carbon monoxide, methanol, acetate, methylated amines, short-chained alcohols, and methyl mercaptan. Although there is one known methanogenic species that can utilize up to seven substrates most other methanogens are highly specialized and are capable of metabolizing only one or two substrates. Therefore, other bacterial species are required to biodegrade the complex organic matter into Simple organic compounds for the methanogens.

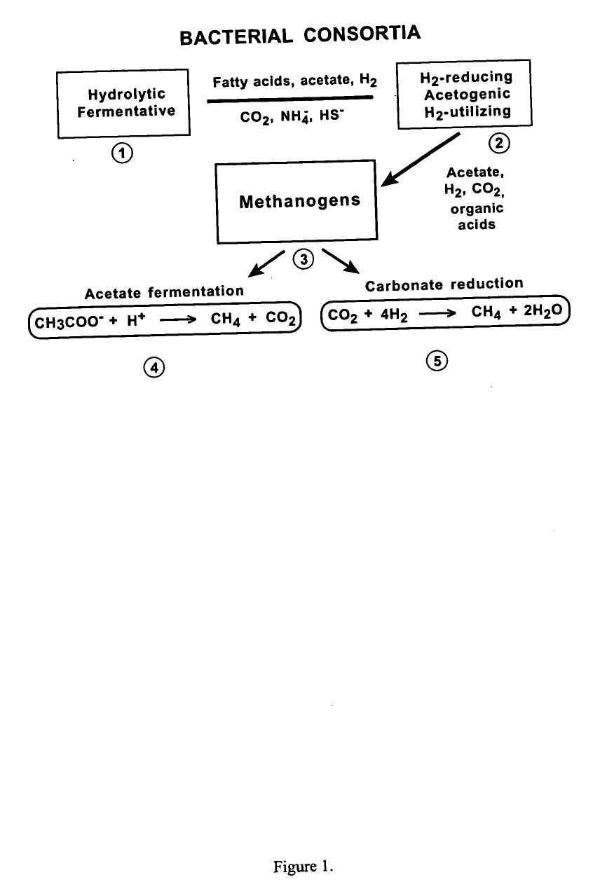

The bioconversion of organic matter to methane and other organic compounds by anaerobic microorganisms typically consists of a three—step process involving different major groups of microorganisms (See FIG. 1). During the initial step, complex organic molecules and polymers are transformed into organic acids and alcohols including propionate butyrate, valerate, lactate, formate, ethanol, and formate by hydrolytic and fermentative bacteria 1 which are also called acid farmers. These bacteria are mostly obligate anaerobes, although some facultative anaerobes are probably present as well. A second group of bacterial consortia collectively called obligate, hydrogen-producing acetogenic bacteria 2 convert these organic compounds to acetate, hydrogen, carbon dioxide, and other simple carbon compounds. Homoacetogens that form acetate as the sole reduced end product from certain substrates may also be present. The third step involves the bacteria or introduced into the subsurface, the preferred metabolic pathways used by the methanogens are carbonate reduction 4 and acetate fermentation 5. (See FIG. 1) During carbonate reduction, 75% of the hydrogen atoms in the methane are derived from formation water and bicarbonate ions and, in addition to carbon dioxide, are involved in the metabolic processes. Carbon dioxide and/or bicarbonate are used in intermediary steps such that the net chemical reaction involving two or more bacterial species may result in a net gain or loss of carbon dioxide (bicarbonate ions) from the system. Acetate fermentation involves the reduction of acetate or related organic compounds by methanogenic bacteria. Unlike carbonate reduction, the hydrogen and methane generated through acetate fermentation are derived primarily from the methyl group, and only 25% of the hydrogen is obtained from the formation water. Theoretical calculations for waste digestion suggest that during carbohydrate fermentation two-thirds of the methane would be derived from acetate fermentation, whereas one-third from carbonate reduction. However, there are several lines of evidence that indicate carbonate reduction is the preferred metabolic pathway in subsurface coal beds and other organic-rich sediments.

The synergy among various species plays a critical role in the in situ bioconversion of organic matter into methane and other compounds. The consumption of hydrogen generated from other microorganisms by methanogens creates thermodynamically favorable conditions for the catabolism of many alcohols, fatty acids, and aromatic compounds. It is the removal of hydrogen that makes the bioconversion of these compounds possible. The removal of acetic and other organic acids by methanogens prevents the formation of toxic compounds and acidic conditions—that inhibit the growth of, or are toxic to, all trophic levels in the substrate. Additionally, the methanogens may produce compounds that are stimulatory to other microorganisms in the consortia.

In the absence of methanogens and other microorganisms that consume hydrogen, the bioconversion of coal into hydrogen is possible. Fermentative hydrogen producers, that may be facultative or obligate anaerobes, are reported in many natural environments including the nutrient-poor Sargasso Sea and from flowers. There are reports of hydrogen gases being produced from coal beds suggesting that undiscovered fermentative hydrogen produces are present in some coal beds and possibly in other organic-rich substrates. Fermentative hydrogen producers such as these can be collected and added to the bacterial consortia injected into the subsurface to increase hydrogen production, which in the presence of methanogens, will result in increased methane generation.

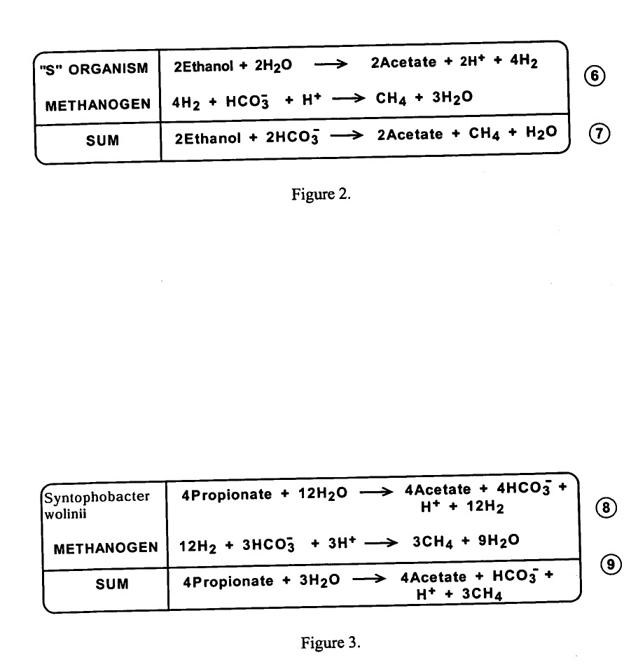

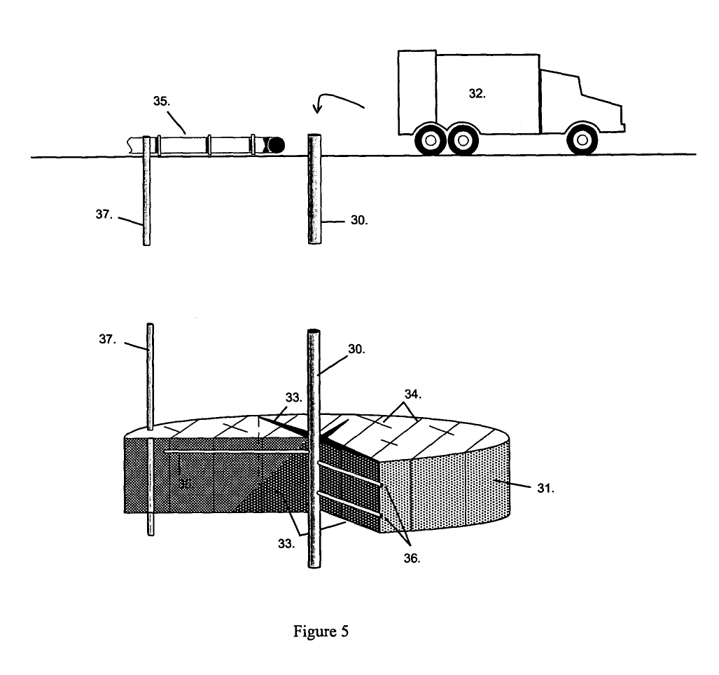

The synergism among various bacterial species in the consortia will ultimately determine the types and quantities of products generated through in situ bioconversion. For example, carbon dioxide is not necessarily consumed in an overall reaction involving multiple bacterial species. See 5 in FIG. 1 and 5, 6, 7, 8, 9 in FIGS. 2 and 3. Carbon dioxide/bicarbonate is used in intermediary steps 6 and 8 such that the net biochemical reaction involving two or more bacterial species may result in the removal of carbon dioxide, as at 7, from the system rather than a net gain of carbon dioxide (bicarbonate ions), as at 9. Therefore, Waste gases such as carbon dioxide and carbon monoxide sequestered into coal beds are food sources that are converted into methane and other organic compounds using the appropriate bacterial consortia and nutrients. Environmental factors such as vitamins, nutrients, trace elements, pH, nitrogen sources, and salinity will be monitored and adjusted to maximize bioconversion. many types bacterial species comprise a consortia, thereby forming a dynamic bacterial community Although dominant bacterial species within the major groups of bacteria may change over time, the relative proportions of the major groups will remain relatively constant, indicating that even dynamic communities may maintain a stable ecosystem function.

The bacterial consortia used in the in situ bioconversion of organic matter described in this invention can be derived from three possible sources and/or a combination of courses. The sources of the bacterial consortia may include: (1) commercial entities which supply known species of bacteria, (2) undiscovered bacterial species which may be obtained from underground coal beds and/or shales; these species have probably adapted genetically to efficiently metabolize the organic matter, and (3) genetically engineered bacterial species or consortia highly adapted to convert organic compounds into methane, the genetic material for these species may be obtained from known bacterial species and those discovered in subsurface environments. Any combination of bacteria from these three sources can be used to generate or enhance methane generation in the subsurface. A detailed description of each of these bacterial sources is described below.

Known bacterial species obtained from any number of commercial firms can be injected into naturally occurring or induced fracture (or cleat) systems of coal, carbonaceous shale, or other organic-rich sediments along with nutrients and trace elements to stimulate bioconversion of the organic matter. In some cases, coal samples from individual coal beds can be tested with commercial bacterial consortia to determine the most efficient Combination of bacteria groups and nutrients. Coal is a complex organic substance that is comprised of several groups of macerals, or major organic matter types, that accumulate in different types of depositional settings such as peat swamps or marshes. Maceral composition, and therefore coal composition, changes laterally and vertically within individual coal beds. Different bacterial consortia and nutrients may work better on specific -maceral groups and therefore, each coal bed may be unique in what types of consortia are most efficient at the in situ bioconversion of the coal.

A second source of bacterial consortia are naturally occurring bacteria 1-hat are associated with coal and other organic-rich sediments in the subsurface. over time, these bacterial species may have become very efficient at metabolizing organic matter in the subsurface through the process of natural selection. The relatively quick adaption of bacteria to local environmental conditions suggests that consortia collected from basins, or individual coal seams, may be genetically unique. Once collected, these bacteria can be grown in laboratory cultures to evaluate and determine factors enhancing and/or limiting the conversion of coal into methane. Relatively little research has been performed on the composition of subsurface bacterial consortia, indicating that there are a significant number of bacterial species that have yet to be discovered. In some cases, a key nutrient or trace element may be missing, and addition of this limiting factor may significantly increase methane production. When bacteria are deprived of nutrients physiological changes occur, and if the state of starvation continues, all metabolic systems cease to function and the bacteria undergo metabolic arrest. When environmental conditions change, the bacteria may recover and establish a viable population again. Therefore, it is possible that some bacteria in organic-rich sediments have reached a state of metabolic arrest and the addition of nutrients is all that is required to activate the population under the present invention. Bacteria from sediments more than one million years old have been successfully revived, indicating that these bacteria can be utilized in the bioconversion process.

Anaerobic bacteria from the subsurface can be collected by several different methods that include (1) produced or sampled formation water, (2) drill cuttings, (3) sidewall core samples (4) whole core-samples, and (5) pressurized whole core samples. Pressurized core samples present the best opportunity to collect viable consortia, whereas collection of bacteria from formation waters may result in collection of only a few species rather than a representative sample of the bacterial consortia. Drill cuttings, sidewall cores and whole cores may yield viable bacteria on the inner parts of the sample, but collection of representative consortia is a concern. Methanogens are obligate anaerobes, but can remain viable in the presence of oxygen for as much as 24 hours by forming multicellular lumps. Additionally, anoxic/reducing microenvironments in an oxygenated system can potentially extend anaerobic bacterial viability longer. In some cases, drill cuttings collected and placed in anaerobic sealed containers will contain bacteria that are capable of converting the coal to methane within a few hours, thereby giving erroneous gas content measurements.

The third source of bacteria consortia for injection into the subsurface are genetically altered bacteria from the laboratory. With the progressive development of genetic engineering technology, biologists are now capable of genetically engineering microorganisms to have abilities beyond their “normal” capacities. Special bacterial species that are adapted over time in the, laboratory to efficiently metabolize coal and other organic-rich substrates and/or genetically-engineered bacteria, along with appropriate nutrients, when injected into the subsurface may enhance the bioconversion of organic matter. The original genetic material for genetic engineering may be derived from unique (known and yet to be discovered:) subsurface bacterial species. The genetically engineered species can be added to other bacterial species that include both commercial and noncommercial bacteria and injected into the subsurface.

The final source of bacterial consortia for the present invention represents a combination of the three other sources described above; commercial bacterial consortia, genetically unique bacterial obtained from the subsurface, and genetically engineered bacteria. A combination of two or all three sources may prove to be the most efficient means of identifying and promoting the in situ bio-conversion of organic matter into methane. Regardless of the source of the bacterial consortia, once injected into the subsurface, the bacterial species in the consortia will adjust themselves according to existing environmental factors to promote the bioconversion of organic matter. Addition of different bacterial species or bacterial groups and/or specific nutrients probably will be required to promote bioconversion as well.

The present invention includes several methodologies and techniques maximizing the rate of bioconversion in the subsurface. Limiting factors for in situ bioconversion are the injection of inappropriate bacterial consortia and/or nutrients, inadequate access of bacteria and nutrients into the natural or artificially induced fracture or cleat systems, insufficient fracture surface area to maximize gas generation, and the accumulation of toxic waste products over time. In coal beds, bacteria will metabolize the organic matter along fracture or cleat surfaces, and inside larger pores within the coal matrix, whereas in fine-grained carbonaceous shales and organic-rich shales, the bacteria will utilize organic matter dispersed within the silty and clay-size sediments. Regardless of the type of organic-rich material that serves as the bioconversion substrate, the bacteria and nutrients in this invention will be injected into the reservoir through naturally and/or artificial-occurring fractures. The fracture, pore, and grain surface areas available for bioconversion will largely determine in situ bioconversion rates and the ultimate yield of methane and other compounds from the organic matter in this art, Therefore, the methods used for assuring maximum bacterial access in the reservoir through naturally-occurring fracture systems is an important part of this invention.

There are several methods or combination of injection techniques that can be utilized to assure that when the bacteria and nutrients are injected into the fractures they access the largest part of the reservoir as possible, and therefore, assure maximum bioconversion rates. The present invention recognizes that fracture orientation, present-day in situ stress direction, reservoir (coal and/or shale) geometry, and local structure must be taken into consideration when injecting bacteria and nutrients into the organic-rich reservoir. For example, there are two major networks (called cleats) in coal beds, termed the f ace cleat and butt cleat system. The face cleats are often more laterally continuous and permeable, whereas the butt cleats (which form abutting relationships with the face cleats) are less continuous and permeable. During the stimulation of coal bed methane wells, the induced fractures intersect the primary face cleats that allow greater access to the reservoir. However, when the present-day in situ stress direction is perpendicular the face cleats, then stress pressure closes the face cleats thereby reducing permeability, but at the same time in situ pressures increase permeability of the butt cleats system. Under these conditions, induced fractures are perpendicular to the butt cleat direction, providing better access to the natural fracture system in the reservoir. The geometry of the injection and producing wells, and whether or not horizontal cells are used to access the reservoir, depend largely upon local geologic and hydrologic condition.

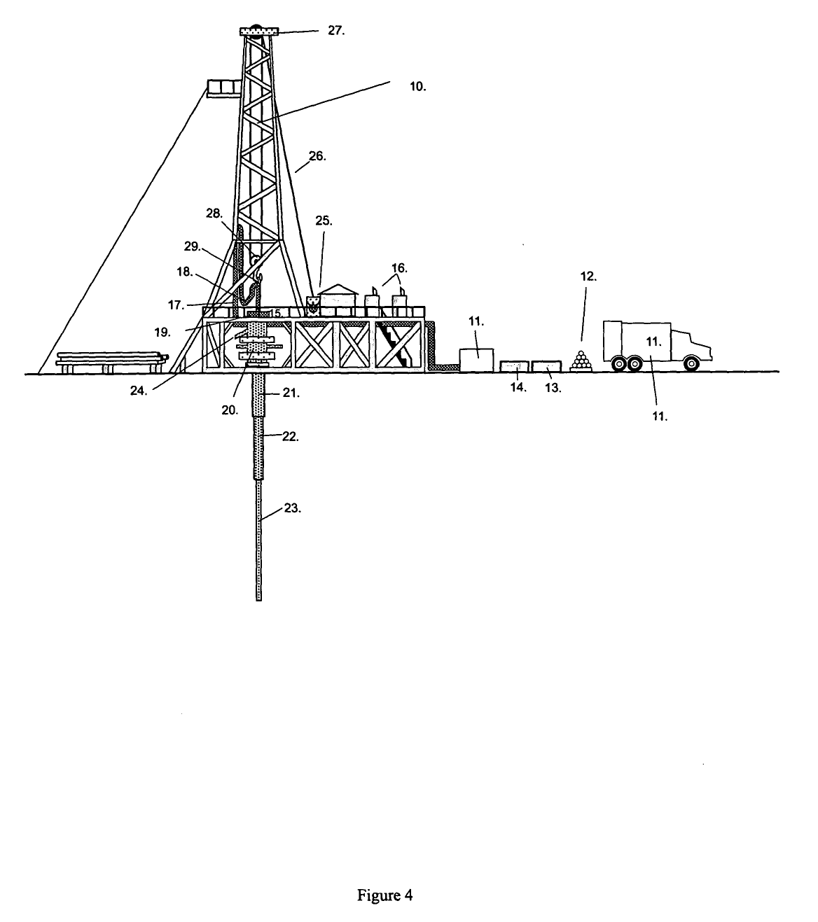

The objective of hydraulic fracture stimulation of coal bed methane, as in conventional oil and gas wells, is to generate an induced fracture network that connects with the naturally occurring fracture network of the reservoir. In the present invention, bacteria a)-id/c)r nutrients are injected into the naturally-occurring and artificially-induced fractures under pressure to drive the mixture into naturally-occurring fractures deep into the reservoir to maximize bioconversion rates and efficiency. During fracture stimulation of reservoirs, as illustrated in FIG. 4, sand propant and various chemicals are pumped into the formation under high pressure through a drill rig 10, mobile trucks 11 and surface equipment into the reservoir. During drilling operation, chemicals and/or nutrients 12 are mixed in mixing pits 14 with mud from a mud pit 13 and injected into the reservoir using a pump 15 powered by diesel engines 16. The mixture passes through the standpipe 17 and flexible Kelly hose 18, through the Kelly bushing 19 and blowout preventers 20, into the well bore. Several type Of Casing, including surface 21, intermediate 22, and production 23, are required to isolate the drilling fluids from fresh water aquifers and to provide well bore stability. Mud and rock cuttings from the subsurface are returned to the mudpits via the mud return line 24. Pipe and casing are lowered and raised from the well bore using drawworks 25 and the hoisting cable 26 that passes over the crown block 27 and to the traveling block 28, the swivel 29 allows the drill pipe to turn freely.

Bacteria and nutrients 11 may be injected into the reservoir at the same time as fracture stimulation and/or after the hydraulic fractures are generated. Most in situ microbial applications are expected to occur after fracture stimulation and removal of completion fluids when subsurface anaerobic conditions are reestablished. However, under simultaneous in situ microbial and fracture stimulation, the use of stimulation fluids under anoxic or suboxic conditions is preferred so that anaerobic conditions in the reservoir are maintained, or can be readily attained after stimulation. The injection of aerobic bacteria during simultaneous stimulation would result in the rapid consumption of oxygen and return to anaerobic conditions.

During fracture stimulation and simultaneous in situ microbially enhanced stimulation of reservoirs, sand propant and various chemicals may be pumped down the well bore 30 under high pressure into the coal/organic-rich reservoir 31 from mobile trucks 32 and/or equipment on the surface. See FIG. 5. Artificially induced fractures 33 interest the naturally occurring fracture system 34 thereby allowing greater access to the reservoir and improving production. Bacteria and nutrients may or may not be included with the fracture fluids depending on the type of reservoir as well as local environmental conditions and the stability of the consortia utilized. in some cases, pretreatment fluids that modify the coal, carbonaceous shale, or organic-rich shale for bioconversion may be used with the fracture fluids. However, the preferred method for encouraging in situ bioconversion of organic matter is to inject bacteria and nutrients under pressure and anaerobic conditions after hydraulic fracture stimulation and subsequent flushing of the well. The ‘mixture of bacterial consortia, nutrients, and water can be prepared at the well site or transported to the well using vehicles.

Carbon dioxide and carbon monoxide that are initially sequestered in coal beds to serve as a food source for the bacterial consortia can be brought to the well site by a pipeline system 35 or transported under pressure in trucks. An alternative for accessing the reservoir that is used with or without fracture stimulation, is the application of laterals formed roughly parallel (horizontal) to the tops and bottoms of coal, carbonaceous shale, or organic-rich shale. These laterals 36 are Either drilled outwardly from the main well bore 30 or are generated through high-pressure water technology. High-pressure water jet technology is used to drill laterals through consolidated sediments and, therefore would work well in coal and many other organic-rich sediments that are more friable. Horizontally-drilled and/or water-jet laterals may extend hundreds or thousands of feet from the main well bore, and therefore, provide much better access to the reservoir for bioconversion than conventional hydraulic stimulations which do not extend as far into the reservoir. This method is unique in that it uses the laterals 36 primarily as a means to inject fluids into the reservoir rather than simply using the laterals to extract gas or other hydrocarbons.

Once access to the reservoir 31 is established, multiple injections of bacteria and nutrients that may include different species or groups of bacteria and/or various mixtures of nutrients may be injected into the well bore over a period of days, weeks, months, or years to promote stabilization of the subsurface consortia and optimize the in situ bioconversion of organic matter. In the case of carbon dioxide and carbon monoxide sequestion, injection of these gases followed by bacteria and/or nutrients and subsequent conversion to methane is repeated until the subsurface coal can no longer support bacterial activity at acceptable levels. Successful access to the reservoir, in situ bioconversion efficiency, and determination of the quantity of methane and other compounds generated during the process can be evaluated from small diameter observation wells 37 strategically placed near the main well (or wells) to collect samples over time.

Following the hydraulic fracture stimulation and injection of bacteria and nutrients into the subsurface reservoir, the well J5 then shut in for a period of time to allow the bacterial consortia to stabilize, thereby encouraging the bioconversion of the organic matter to methane and other compounds. After a brief stabilization time, the water and gases produced through the in situ bioconversion of the organic matter can be produced through the well bore 30 along with gases already present in the reservoir 31. in this came, the injection and producing well are the same and the process is a single well bioconversion. One drawback to this methodology is that toxic waste products may build up in the reservoir thereby inhibiting the bioconversion process.

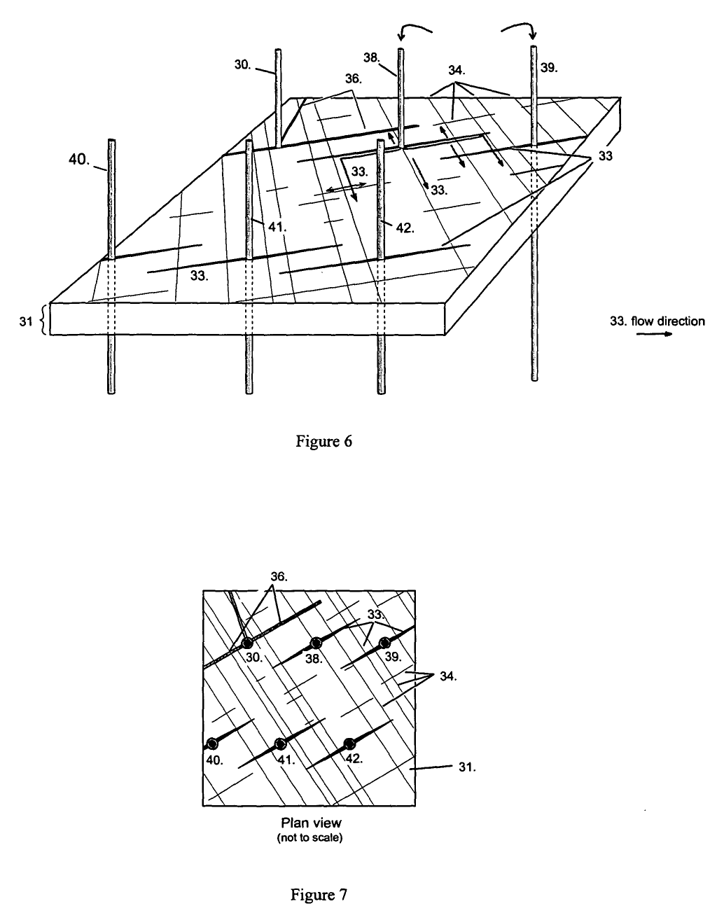

In alternative methods of this invention (see FIGS. 6 and 7) additional mixtures of bacterial consortia and water may be injected into the well bore 30, or series of well bores 30, 39, 39 following the stabilization period, thereby providing energy to continue to drive the bacterial consortia and nutrients deeper into the reservoir. This process is called multi-well bioconversion. Many bacteria are capable of moving through fractures under their own energy and/or with formation waters migrating through the fracture network 33, 34. However, continued injection under pressure will be more efficient at moving the bacterial consortia and nutrients to deeper parts of the reservoir in a more time efficient manner. One benefit of the methodology of using repeated injections is the removal of toxic waste products and introduction of fresh nutrients to the bacterial consortia. The injected fluids will tend to move through the larger and more permeable fracture system that are often the most continuous However, once the bacterial have entered deep into the reservoir via these natural fracture systems, the injected bacteria will then migrate under their own energy to smaller fractures and microfractures in the coal and organic-rich sediment, thereby accessing a volumetrically mush larger part of the reservoir. The injected fluids, methane and other organic compounds formed from the in situ bioconversion of the organic matter are removed from the reservoir by a series of recovery wells 40, 41, 42. The recovery wells 40, 41, 42 may be hydraulically-stimulated and/or have laterals extending from the main well bore in the manner of laterals 36 of well bore 30 as described with reference to FIG. 5 so that the natural fracture system is adequately accessed for the efficient recovery of methane.

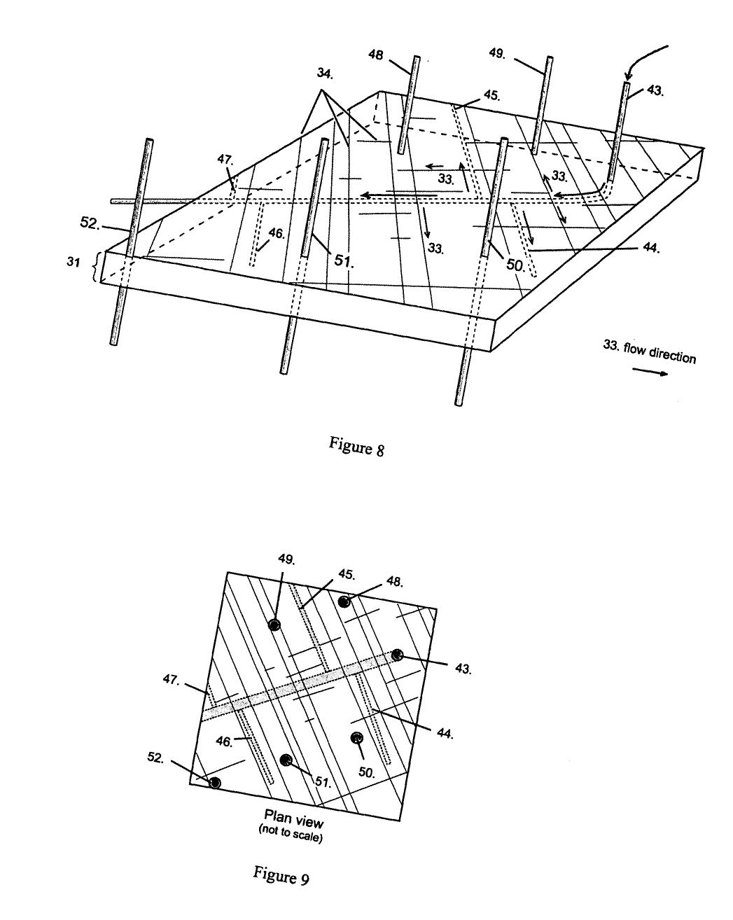

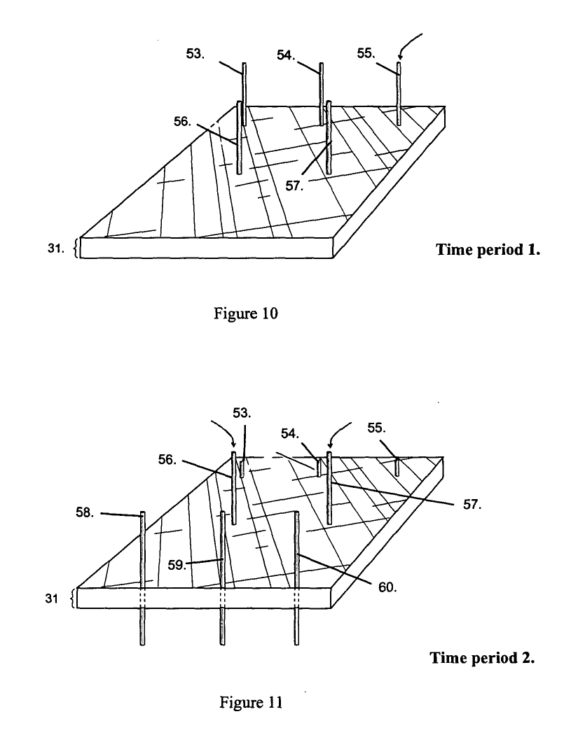

This invention also encompasses the use of horizontal wells, including laterals from these wells, for the injection of bacteria and nutrients into the organic-rich reservoir. During horizontal injection well bioconversion a horizontal injection well 43, as shown in FIGS. 8 and 9, is drilled, first vertically to the formation 31, then perpendicular to the major trends of naturally occurring fractures. or cleats in the coal bed or other organic-rich reservoir 31, to maximize the injection distances for bacterial consortia and/or nutrients as well as potentially increasing the bioconversion rate. If the in situ stress direction is perpendicular to the face cleat orientation (major fracture trends), then the horizontal well may he drilled parallel to the face cleats direction (perpendicular to butt cleats). In some cases a series of lateral holes 44, 45, 46, 47, that are roughly parallel to the top and bottom of the coal bed or organic rich sediment, will be drilled off the main horizontal well 43 to provide better access to the reservoir. Alternatively, pressurized water-jet technology may be used to drill the laterals 44-47 off of the main horizontal well 50 or be utilized to create the horizontal well itself.

Regardless of how the horizontal well 50 and laterals 51-54 are created, by drilling or water jet technology, the primary purpose of the holes 43-47 is for the injection of bacteria and/or nutrients for in situ bioconversion. Depending on reservoir geometry, pressure and other factors, recovery wells 48, 49, 50, 51, 52 may or may not be hydraulically fractured or stimulated in order to increase production of methane and other compounds from the in situ bioconversion of organic matter. A horizontal well system as just described for the present invention would also be very efficient at injecting and sequestering carbon dioxide and carbon monoxide in coal beds. As coal beds adsorb these gases, the coal swells and reduces permeability. More carbon dioxide and carbon monoxide could be sequestered using this technique because the horizontal wells would provide better access to the reservoir. The deep sequestration of carbon dioxide and carbon monoxide followed by injection of bacteria and nutrients would result in more efficient in situ bioconversion, because of the greater reservoir volume accessed by the carbon dioxide and bacteria.

The geometry of injector and recovery wells can be variable, but must be based on local geologic, structural, and hydrologic conditions in order to maximize the injection distances of the bacteria and/or nutrients and to attain maximum recovery of methane. Additionally, injector well geometry will maximize the amount of carbon dioxide and carbon monoxide sequestered into the coal beds, and therefore, the amount of food available for the in situ bioconversion process. The present invention may also utilize a dynamic injector and recovery well bioconversion system in which various sections of a coal bed and/or other organic-rich sediment are sequentially injected with bacteria and nutrients. For example, as shown in FIG. 1C), the process may begin with the injection of bacteria and/or nutrients into injection wells 53, 54, 55 followed by the production of methane and other organic compounds from the recovery wells 56, 57. At some point in time, the coal and/or organic matter between the injectors and recovery wells may become less biodegradable as the easily consumed organic compounds are metabolized during the bioconversion process. Under these conditions, the recovery wells 56, 57 are converted into new injector wells and a new series of recovery wells 58, 59, 60 (see FIG. 11) are drilled in proximity to the converted wells 56, 57. The original injector wells 53, 54, 55 may be plugged and abandoned or used for the sequestration of carbon dioxide without subsequent bioconversion, Alternatively, they may be used to dispose of water from ongoing operations. The process of reservoir stimulation is followed by injection, bioconversion, and recovery as described above, thereby producing methane and other gases from a different part of the reservoir 31. This embodiment of the invention represents an effective, systematic, and controlled means for biologically mining the organic-rich material in the reservoir 31 through a series of injector and recovery wells. This method of the invention ‘may also be modified to use a combination of techniques previously described, including horizontal wells for injection and/or recovery using the dynamic injector and recovery well system instead of vertical wells.

The following examples serve to illustrate the practical application of the present invention. It should be noted that any combination of technologies discussed in one of these examples is also applicable to other bioconversion examples or situations as well, For example, the horizontal injection well bioconversion process can be applied to the bioconversion of sequestered carbon dioxide. Dynamic injector and recovery well bioconversion is applicable to any of the processes. The type of bioconversion process employed will depend on the hydrogeological characteristics of the project areas as well as economic considerations of the project.

### EXAMPLE 1

Coal geometry and present-day in situ stress direction favor the application of horizontal injection well bioconversion. The subbituminous-rank coal seems already contain secondary biogenic gases based on isotopic analysis of gas samples and low gas contents. A horizontal well with laterals, such as shown in FIGS. a and 9, may be drilled and coal cuttings collected to analyze the types of bacteria that are present in the reservoir. Following completion and swabbing of the well, a bacterial consortia obtained from a commercial firm may be injected under pressure along with molasses and other nutrients. Monitoring wells would indicate that methane and carbon dioxide are being generated from the coal beds and that gas contents are increasing slightly. Hole core and water samples may be collected from monitoring wells and sent to the laboratory for comparison with the bacterial consortia samples obtained when drilling the horizontal well. Coal maceral analyses may be performed to determine the dominant type of organic matter present in the coal sample.

A modified mixture of bacteria and nutrients may be injected into the reservoir fracture system through the horizontal well and lateral system, and the bioconversion rate verified by additional monitor wells. New analyses would indicate significant bioconversion activity and recovery wells may be drilled downdip of the horizontal well to collect methane generated during the bioconversion of the coal. Because the horizontal well and laterals efficiently intersect the naturally occurring fractures arid cleat; in the reservoir, it could be decided that infill drilling within the lateral system is not required. New analyses would indicate that gas contents and methane concentration are increasing, indicating that the bacteria consortia have attained a stabilized community. Analyses of coal ‘macerals obtained during and after in situ bioconversion would verify the types of organic matter undergoing bioconversion and this information would be used to more efficiently select drill sites and to select more efficient bacterial consortia based on a coal depositional model, a model of the type of depositional environments in which the coal form indicates the types and lateral extent of plant communities associated with fresh and salt water marshes and swamps.

### EXAMPLE 2

Shale samples and water samples from a test well drilled through a thick, fractured, organic-rich shale would be analyzed for total organic carbon (TOC) and maceral analyses and evaluated for the presence of secondary biogenic gases and bacteria. The thermal maturity and type of organic mater (in this example, Type I or lacustrine/lake) and the presence of an acceptable fracture network would indicate that bioconversion of the shale is possible. The relatively shallow depths, and great thickness of the shale indicate that only a relatively small percentage of the organic matter contained in the shale needs to be converted into methane to make the enhanced shale recovery economically viable.

Based on laboratory and detailed electric log correlations that delineate the lateral and vertical extent of high in situ TOC contents, a series of wells would be drilled and fracture stimulated Bacteria and nutrients would be injected under pressure to transport the bacteria deep into the fracture system.

A series of adjacent wells would be drilled to collect ‘methane generated during the bioconversion process, whereas formation water produced with biogenic gases would be treated and reinjected into the reservoir along with a modified mixture of bacteria and nutrients to enhance the bioconversion rate. After a period of time, when bioconversion rates decrease, it could be decided to convert the project into a dynamic injector and recovery well bioconversion system (such as described with reference to FIGS. 10 and 11) and the process would be repeated.

### EXAMPLE 3

Carbon dioxide and carbon monoxide removed from the waste stream of coal-powered electric plants would be transported to the bioconversion site via a pipeline system and injected into highly permeable coal beds located near a coal mine and power generation station. The bioconversion site would be located based on economic considerations and the coal beds in which the carbon dioxide and carbon monoxide are to be injected. Production wells would be drilled downdip and along strike from injector wells.

A carefully selected bacterial consortia, that includes commercial bacteria, naturally occurring bacteria from the coal reservoir, and genetically engineered bacteria, as well as nutrients could be injected into the coal beds following the sequestration of carbon dioxide and carbon monoxide. The bacteria and nutrient mixture would be injected into the naturally occurring fracture and cleat system following hydraulic fracturing of the wells. In this example, periodic pressure injection would assure that the bacteria and nutrients are continually forced deep into the reservoir where they can access and convert the carbon dioxide into methane. The bacteria consortia and nutrients are monitored and adjusted so that the net bioconversion reaction consumes carbon dioxide, carbon monoxide and bicarbonate as well as organic compounds on the coal to generate methane. When bioconversion rates declines and laboratory analysis of coal, water, and gas samples indicate that the majority of the injected gases have already been converted into methane, the producing wells would be temporarily shut in. Carbon dioxide and carbon monoxide would be reinjected into the coals and the process repeated. Formation waters produced from the coal beds during this process are preferably treated with bacteria and nutrients and reinjected into existing injection wells and/or transported to an adjacent project area where! the same bioconversion process may be applied.

### EXAMPLE 4

Hydrogen-rich Subbitumincus to high-volatile A bituminous coal beds containing appreciable quantities of paraffins (waxes) and wet gases are produced during a coalification process. The combination of lower reservoir permeability due to paraffin generation during coalification and excessive wax production during coal gas production makes the coalbed methane project marginally economic-al. A combination of horizontal injection well bioconversion and multiwell bioconversion techniques may be employed to maximize bacterial access to the reservoir and to reduce excessive wax production. No viable bacterial consortia are present in the coal samples, so a combination of commercial bacteria, genetically engineered bacteria, and naturally occurring bacteria collected from the same formation outside the project area as well as nutrients would be injected into the wells. The bacteria preferentially metabolize the long-chained n-alkanes that comprise the waxy paraffins, thereby reducing or eliminating excessive wax production and generating significant quantities of methane during bioconversion. once permeability and reservoir access have improved in the area, a dynamic injector and recovery well bioconversion process ‘may be employed to maximize bioconversion rates and the quantity of methane generated. As with previous examples, bioconversion would be continually monitored and the bacteria consortia and nutrients adjusted to maximize the amount of methane produced from the organic matter.

Several methods of recovering gas from subsurface formations of coal, carbonaceous shale and organic-rich shales, by injection of consortia of selected biological microorganisms, have been described herein. Specific examples have been described. many variations of the invention can )De practiced by those skilled in the art without departing from the spirit of the invention. Accordingly, the invention is limited only by the claims which follow.

1. A method of generating and recovering gas from naturally existing Subsurface formations of coal, carbonaceous shale or organic; rich shales comprising the steps of:
injecting into fractures of said subsurface formation, under substantially anaerobic conditions, a consortia of selected anaerobic biological microorganisms for in situ conversion of organic compounds in said formation into methane and other compounds; and
producing said methane through at least one recovery well extending from said subsurface formation to the surface.

2. The method of generating and recovering gas as set forth in claim 1 in which said consortia of selected anaerobic biological microorganisms include methanogens.

3. The method of generating and recovering gas as set forth in claim 2 in which said consortia of selected anaerobic biological organisms includes at least three groups of organisms: an acid former group for transforming complex organic molecules into 5 organic acids and alcohols by hydrolysis and fermentation; an obligate, hydrogen producing acetogenic group for converting said organic acids and alcohols to hydrogen and single carbon compounds; and a group comprising said -methanogens for converting said hydrogen and said simple carbon compounds into said methane.

4. The method of generating and recovering gas as set forth in claim 3 in which said methanogens convert said hydrogen and said simple carbon compounds into said methane by acetate fermentation or carbonate reduction.

5. The method of generating and recovering gas as set forth in claim 3 in which said three groups of microorganisms may be selected from one or more of the following consortia Of microorganisms: commercially available bacteria; genetically unique bacteria obtained from said subsurface formation and; genetically unique, laboratory altered bacteria.

6. The method of generating and recovering gas as set forth in claim 1 including the injection of nutrients into said fractures of said subsurface formation to feed said consortia of selected anaerobic !biological microorganisms.

7. The method of generating and recovering gas as set forth in claim 6 in which said nutrients comprise at least one of the following: carbon dioxide and carbon monoxide.

8. The method of generating and recovering gas as set forth in claim 7 in which said carbon dioxide and/or said carbon monoxide is injected into and sequestered in said subsurface formation for a period of time prior to said injection of said consortia of selected anaerobic biological organisms.

9. A method of recovering gas from naturally existing subsurface formations of coal, carbonaceous shale or organic rich shales iii which are contained preexisting consortia of substantially inactive anaerobic biological microorganisms comprising the steps of:
injecting bacterial nutrients into fractures of said subsurface formation, under substantially anaerobic conditions, to feed and activate said inactive biological-microorganisms for in situ conversion of organic compounds in said formation into methane and other compounds; and
producing said methane through at least one well extending from said subsurface formation to the surface.

10. The method of generating and recovering gas as set forth in claim 9 in which said bacterial nutrients comprise at least one of the following: carbon dioxide and carbon monoxide.

11. The method of generating and recovering gas as set forth in claim 9 in which a consortia of additional selected biological organisms are also injected into said fractures of said subsurface formation to aid in said in situ conversion of organic compounds into said methane and other compounds.

12. The method of generating and recovering gas as set forth in claim 11 in which carbon dioxide and/or said carbon monoxide is injected into and sequestered in said subsurface formation for a period of time prior to injection of said additional selected biological organisms.

13. A method of generating and recovering gas from naturally existing subsurface formations of coal, carbonaceous shale or organic rich shales comprising the steps of:
drilling a well into said subsurface formation;
injecting into fractures of said subsurface formation, through said well, a consortia of selected anaerobic biological microorganisms for in situ conversion of organic compounds in said formation into methane and other compounds shutting in said well for a determined period of time; and
opening said well and producing said methane through said well,

14. The method of generating and recovering gas as set forth in claim 13 in which said subsurface formation is artificially fractured after drilling thereof to provide additional fractures therein.

15. The method of generating and recovering gas as set forth in claim 14 in which at least some of said consortia of selected anaerobic biological microorganisms are injected simultaneously with injection of fracturing fluids for said artificial fracturing of said subsurface formation.

16. The method of generating and recovering gas as set forth in claim 13 in which water and bacterial nutrients are injected with said consortia of selected biological microorganisms into said fractures of said subsurface formation.

17. The method of generating and recovering gas as set forth in claim 13 in which bacterial nutrients are injected, through said well, into said fractures of said subsurface formation to feed said consortia of selected anaerobic biological organisms.

18. The method of generating and recovering gas as set forth in claim 17 in which said nutrients comprise at least one of the following compounds: carbon dioxide and carbon monoxide.

19. The method of generating and recovering gas as set forth in claim 18 in which said carbon dioxide and/or carbon monoxide and said consortia of selected anaerobic biological organisms are repeatedly and alternately injected over a period of time until bacterial activity in said conversion of said organic Compounds to said methane and other compounds reduced below acceptable levels.

20. The method of generating and recovering gas as set forth in claim 13 in which said drilling of said well provides a main bore, said method also comprising the additional step of forming one or more laterals extending outwardly from said main bore into other regions of said subsurface formation and into which said consortia :)f selected anaerobic biological microorganisms are also injected.

21. The method of generating and recovering gas as set forth in claim 13 in which said subsurface formation is artificially fractured after the forming of said one or more laterals to provide additional fractures therein.

22. The method of generating and recovering gas as set forth in claim 21 in which at least some of said selected consortia of selected anaerobic biological microorganisms are injected simultaneously with fracturing fluids for said artificial fracturing of said subsurface formation.

23. A method of generating and recovering gas from naturally existing subsurface formations of coal, carbonaceous shale or organic rich shales comprising the steps of:
drilling at least one injection well into said subsurface formation;
drilling at least one recovery well into said subsurface formation;
injecting into fractures of said subsurface formation, through said injection well, a consortia of selected anaerobic biological microorganisms for in situ conversion of organic compounds in said formation into methane and other compounds and producing said methane through said recovery well.

24. The method of generating and recovering gas as set forth in claim 23 in which at least one of said injection well and said recovery well are artificially fractured after drilling thereof to provide additional fractures therein.

25. The method of generating and recovering gas as set forth in claim 23 in which said drilling of said injection well provides a main bore, said method also comprising the additional step of forming one or more laterals extending outwardly from said main bore into other regions of said subsurface formation and into which said consortia of selected anaerobic biological microorganisms are also injected.

26. The method of generating and recovering gas as set forth in claim 23 in which said injection well, after a period of time, is shut in, and at least one other recovery well is drilled into said subsurface formation, production through said one recovery well being terminated and said consortia of selected anaerobic biological, microorganisms then being injected into fractures of said subsurface formation through said one recovery well as an injection well for in situ conversion of organic compounds in said formation into methane and other compounds; and producing said methane through said one other recovery well.
